(19) United States
(12) Patent Application Publication
(10) Pub. No.: US 2021/0361920 A1
(43) Pub. Date: Nov. 25, 2021

(54) Drug Delivery Device and System with Metered Dosing for Semi-Solid Compositions

(71) Applicant: Compounding Innovations LLC, Costa Mesa, CA (US)

(72) Inventor: Huong Mai Tran, Huntington Beach, CA (US)

(73) Assignee: Compounding Innovations LLC, Costa Mesa, CA (US)

(21) Appl. No.: 16/878,589

(22) Filed: May 19, 2020

(51) Int. Cl. A61M 35/00; A61M 37/00; A61M 39/20

(52) U.S. Cl. CPC A61M 35/00; A61M 2205/6081; A61M 39/20; A61M 37/00

(57) ABSTRACT

Described herein is a disposable drug delivery device and system (e.g. a hormone replacement drug in the device), and methods for administering precise, metered doses of topical and transdermal drugs to a user's skin. The device comprises: an overcap; a dispensing cap with at least one hole; a barrel that holds the drug; a threaded spindle centered within the barrel, and connected to a platform that moves upward through the barrel to push the drug out of the hole(s); a bi-directional reset dial that is not connected to the platform, to mark the starting-stopping point; and a unidirectional dosing knob that is connected to the platform, with markings for sequential increments of drug dispensing (e.g. 0.1 ml-0.9 ml drug/actuation). Due to the reset dial, a user is not required to keep count of their dosing knob actuations to know when they have dispensed the entire dose.

### FIELD OF THE INVENTION

The present invention relates generally to drug delivery devices and systems for topical and transdermal applications of precise, metered doses of a semi-solid drug, such as one for hormone replacement therapy, which passes through the skin into the patient's systemic circulation. In particular, the device comprises a reset dial and a dosing knob to enable a user to dispense the dose incrementally without having to count actuations of the dosing knob.

### BACKGROUND OF THE INVENTION

The prior art discloses a number of metered drug delivery devices for dispensing semi-solid compositions incrementally, one actuation at a time. For example, for the Topi-Click® device, a user twists a device base to force a small amount of the composition to squeeze out of the top of the device, but the amount is only a fraction of the total dose required. For the Topi-Click®, each actuation is referred to as a “click.” The user can then apply the small amount of the semi-solid composition immediately to their skin or continue to pump out more of the composition until the full dose is extruded, and then apply it topically or transdermally.

The main problem with these types of devices is that the user must mentally count how many actuations, or “clicks,” they have performed to know when the total dose has been dispensed. If they are interrupted, or have difficulty concentrating, this may prove to be very difficult. This would most likely interfere with therapy and could possibly result in a wrong dosage dispensed or medications wasted if the user does not know how much was dispensed due to the lost count.

Metered drug delivery devices can also be very expensive and intended for reuse, which can lead to contamination of the drug.

Current devices may also be colored to represent a specific drug. However, with current devices on the market, if the wrong drug is filled into a device with a different color than intended, the user must discard the entire device.

Thus, there is a need in the marketplace for an economical, disposable drug delivery device and system that easily delivers precise metered doses (e.g. 0.1 ml increments) of semi-solid drugs (e.g. creams, gels, etc.). The devices should serve as both a drug storage unit and a dispenser, as well as an applicator for topical and transdermal applications. The device should also have a reset feature so that a user can simply dispense out a specific volume of the drug without having to count each individual dosing increment.

### SUMMARY OF THE INVENTION

The present invention comprises a disposable drug delivery device and system (e.g. a hormone replacement drug in the device), and methods of use for administering precise, metered doses of topical and transdermal drugs to a user's skin. The device comprises: an overcap to prevent contamination and evaporation of the drug; a dispensing cap with at least one hole or aperture to extrude the drug; a barrel that holds the drug; a threaded spindle centered within the barrel, and connected to a platform that moves only upward through the barrel to push the drug out of the aperture(s) on the dispensing cap; a bi-directional reset dial that is not connected to the platform; and a uni-directional dosing knob that is connected to the platform, with each actuation of it causing a fixed amount (e.g. unit) of the drug dose to extrude from the dispensing cap.

In an embodiment, the present invention comprises a drug delivery system with the device storing a prescription topical or a transdermal drug. In an exemplary embodiment, the transdermal drug is for human identical hormone replacement therapy (HIHRT); and comprises a transdermal bio-identical hormone replacement cream (i.e. biologically identical to a human produced hormone) that is absorbed through a patient's skin to their systemic circulation.

A main advantage of the device is due to the reset dial, which does not require a user to keep count of their actuations with each turn of the dosing knob to know when they have dispensed the entire dose. The reset dial comprises the word “Reset” and an arrow below it pointing to dosing knob volumetric markings. The reset dial can rotate clockwise and counterclockwise around the barrel and dosing knob. For example, the dosing knob comprises volumetric dosing increments of 0.1 ml, 0.2 ml, etc. up to 1 ml, printed at 10 equidistant locations around the circumference. The dosing knob can only be turned in a clockwise fashion (as viewed from below), with each actuation automatically dispensing a dose unit from the dispensing cap. In most cases, the reset dial is set to 0.0 ml before starting the dose dispensing, and the user knows to stop dispensing when the final total dose's volumetric mark on the dosing knob is aligned with the reset arrow (e.g. a 0.8 ml dose stops the dosing knob with 0.8 ml aligned with the reset arrow).

Various embodiments of the present invention further comprise a method of use of a disposable, drug delivery device for precise, metered dosing of a semi-solid compositions, comprising in step one, providing: a) a hollow barrel with a tubular outer wall and inner wall forming a cavity, wherein the barrel is able to store a plurality of doses of a semi-solid composition within the cavity, and without leakage of the composition from the device; b) a threaded spindle unit, centered vertically within said barrel, comprising: 1) an elevator platform comprising a thin, horizontal, flat disc top side with a centered threaded hole, and 2) a threaded screw passing through the threaded hole; c) a reset dial displaying a reset arrow, the dial positioned below the barrel and above a dosing knob, and able to manually rotate bi-directionally without turning the barrel, the spindle unit, or the dosing knob; d) the dosing knob connected beneath the reset dial, and able to be manually and incrementally rotated unidirectionally per each actuation or turn of the dosing knob; e) a dispensing cap connected to the top of said barrel, comprising at least one aperture (e.g. a centered hole) that is wide enough to release a unit of the composition; and f) an overcap covering the dispensing cap when the device is not in use, able to prevent contamination of the dispensing cap and evaporation of the composition.

The method steps further comprise: 2) aligning a reset dial arrow with a dosing knob zero-milliliter volumetric mark; 3) manually, and incrementally, turning the dosing knob clockwise while holding the barrel in position, wherein each actuation of the dosing knob dispenses one unit of the semi-solid composition dose from the dispensing cap hole(s) by pushing the elevator platform up the threaded spindle; 4) stopping the dosing knob when a pre-determined dose volumetric mark is aligned with the reset arrow indicating an entire dose has been dispensed, wherein the user is not required to keep count of the number of dosing knob actuations; and 5) administering a precise amount of the one or more dose units of the semi-solid composition from the dispensing cap directly onto a user's skin.

When the entire dose is greater than 1 milliliter, then the pre-determined dose volumetric mark (i.e. stopping point) comprises subtracting the whole number from the dose and rotating the dosing knob more than a full rotation (e.g. total dose 2.4 ml minus 2 ml, means rotating dosing knob two full rotations and then stopping at 0.4 ml aligned with the reset dial arrow, for a total of 24 actuations).

An aspect of the present invention is a dosing knob with dosing increments of 0.1 ml, 0.2 ml, etc. and a reset dial that can be set at 10 equally distanced stops around the barrel to deliver precise drug doses directly to user's skin. For drugs with larger doses, the dosing increments may be larger (e.g. 0.2 ml, 0.4 ml, etc.).

Another aspect of the invention is to administer drug doses that have not been contaminated by contact with a user's hand, or by exposure from removing the dispensing cap to refill the device. The drug unit(s) on the top surface of the dispensing cap are directly applied to the user's skin, and the device is not reusable so the dispensing cap is never removed from the device after leaving a pharmacy.

Another aspect of the invention is to provide an economical (e.g. a few dollars), disposable drug delivery device for a one-time administration of a prescription drug, or a one-time filling of the device.

Another aspect of the invention is to provide a means of administering a precise amount of the dose without slippage of the dosing knob, via: 1) the dispensing cap is touched directly to the patient's skin, versus to their hands; and 2) an overcap covers the dispensing cap when the device is not in use to prevent the cap and drug from being contaminated and the drug evaporating. The means of the barrel inner protrusion members fitting inside the dosing knob prevents dosing knob slippage while the drug is extruded from the dispensing cap.

Another aspect of the invention is to mark the drug delivery devices with colored coded dispensing caps, so that if the device is accidentally marked incorrectly, then only the dispensing cap is replaced with one that is correctly colored.

These and other aspects of and advantages with the present invention will become apparent from the following detailed description and from the accompanying drawings.

### BRIEF DESCRIPTION OF THE DRAWINGS

The above, as well as other advantages of the present disclosure will become readily apparent to those skilled in the art from the following detailed description when considered in light of the accompanying drawings.

FIG. 1A is a front elevational view of the drug delivery device comprising a transparent or translucent material in the barrel side walls.

FIG. 1B is a bottom perspective view of the drug delivery device comprising a transparent or translucent material in the barrel side walls.

FIG. 2 is another front elevational view of the drug delivery device showing the barrel as having clear side walls to illustrate the cavity of where the drug is stored, and how the threaded spindle and platform push the drug out of the top of the device.

FIG. 3 is a longitudinal cross-sectional view of the device taken along line 3-3 of FIG. 2.

FIG. 4 is an exploded bottom perspective view of the device.

FIG. 5 is an exploded top perspective view of the device.

FIG. 6 is a magnified view of the reset dial and the dosing knob to illustrate how a user sets the reset dial at 0 ml before starting to administer a dose.

FIG. 7A is an exploded bottom perspective view of the connections between the bottom of the barrel, the reset dial and the dosing knob to illustrate how the dosing knob controls the rotation of the threaded spindle and the platform upward to squeeze the drug out of the device dispensing cap.

FIG. 7B is an exploded top perspective view of the barrel, the reset dial and the dosing knob to illustrate the link between the barrel bottom end and rotation of the reset dial and dosing knob.

FIG. 8 is a horizontal cross-sectional view of the device taken along line 8-8 of FIG. 3.

FIG. 9 is a horizontal cross-sectional view of the device taken along line 9-9 of FIG. 3.

FIG. 10A is an exploded view of the spindle unit illustrating the elevator platform, the threaded spindle, and the bottom connection unit.

FIG. 10B is a top perspective view of the elevator platform illustrating the outer channel for holding drug backflow as the platform rises in the barrel per each actuation, and how this facilitates creating a fluid seal between the platform on the barrel inner wall to block leakage of the drug beneath the platform.

FIG. 11 is a bottom perspective view of the overcap and the dispensing cap.

### DETAILED DESCRIPTION OF EXEMPLARY EMBODIMENTS

The following description is merely exemplary in nature and is not intended to limit the present disclosure, application, or uses. It should be understood that throughout the drawings, corresponding reference numerals indicate like or corresponding parts and features.

### Glossary of Terms

Throughout this specification and in the claims that follow, the following terms are defined with the following meanings, unless explicitly stated otherwise.

As used herein, the term “composition” and “drug” refer to a flowable, semi-solid compound (e.g. a gel, lotion, cream, ointment, etc.) comprising a brand name or generic prescription, or an over-the-counter compound, to treat or prevent a disease, or chronic condition in a human patient.

As used herein, the term “prevention” refers to prophylactic administration to a healthy subject to prevent the development of the conditions mentioned herein. Moreover, the term “prevention” means prophylactic administration to patients being in a pre-stage of the conditions to be treated.

As used herein, the term “prophylactically effective amount” as used herein means the amount of the active compounds in the composition that will elicit the biological or medical response in a patient to prevent the onset of a disease or condition being treated.

As used herein, the term “treatment” is understood the management and care of a patient for the purpose of combating a disease, condition or disorder.

As used herein, the term “therapeutically effective amount” refers to an amount of a drug or a therapeutic agent that will elicit the desired biological and/or medical response of a patient that is being sought.

As used herein, the term “user” includes both the patient that self-administers, and the caregiver that administers the drug, such as a female patient self-administering hormone therapy by transdermally administering a biological agent listed herein.

As used herein, the term “administration of” and or “administering a” composition should be understood to mean providing a semi-solid emulsion composition (e.g. gel, lotion, cream, ointment, etc.) of the invention or a pharmaceutically acceptable salt or ester thereof, or a pro-drug thereof to a subject in need of treatment. The administration of the flowable (but not liquid) composition of the present invention in order to practice the present methods of therapy is carried out by administering a therapeutically effective amount of the compounds in the composition to a subject in need of such treatment or prophylaxis. The need for a prophylactic administration according to the methods of the present invention is determined via the use of well-known risk factors. The effective amount of an individual composition is determined, in the final analysis, by the physician in charge of the case, but depends on factors such as the exact disease to be treated, the severity of the disease and other diseases or conditions from which the patient suffers, topical and transdermal administration, other drugs and treatments which the patient may concomitantly require, and other factors in the physician's judgment, etc.

As used herein, the term “about” or “approximately” refers to +/−10%, preferably +/−10%, or most preferably +/−5% of the stated value or range of values.

As used herein, the term “substantially” refers to the geometric shape in the present invention that one of ordinary skill in the art would readily recognize as being significantly similar to in shape (e.g. greater than 70% similarity).

As used herein, the term “pharmaceutical” and “drug” refer to both a brand name prescription or generic version thereof, or an over-the-counter version, comprising a semi-solid emulsion (e.g. cream, lotion, gel, ointment, etc.) that is easily pushed out of the device top dispensing cap to be topically or transdermally administered to a patient's skin for absorption through the skin into the systemic circulation. See Table 1 for a non-limiting listing of exemplary drugs comprising compounded medication and hormone replacement therapy. In one or more embodiments, the pharmaceutical comprises transdermal bio-identical hormone replacement cream that is absorbed through the skin into the systemic circulation; and wherein it is biologically identical to human produced hormone.

As used herein, the term “dose” refers to amount of the total drug in milliliters that is prescribed for administration to the patient topically or transdermally in one setting (e.g. a prescription may require one or more doses to be administered per day). A dose total amount is broken down into mini-doses, or actuations of the dosing knob, wherein each actuation dispenses one “unit” of the dose, e.g. precisely 0.1 milliliter of the drug with a total dose of 0.8 milliliters (8 actuations). A plurality of units comprise the dose administered in one setting.

As used herein, the term “topically” refers to the administration of a drug in the device herein that is dispensed onto a user's skin at an afflicted site (e.g. applying an antibiotic directly to an infected cut).

As used herein, the term “transdermal” refers to the route of administration of a drug in the device that is dispensed at prescribed locations on a patient's skin, from which the drug is absorbed across the layers of dermis and into the patient's systemic circulation (e.g. transdermal hormone replacement therapy).

As used herein, the term “drug delivery system” refers to the device 10 filled with at least one dose of a semi-solid drug intended for topical or transdermal route of administration. In an embodiment, the system is filled with a prescription drug; and it may be marketed as such. Device 10 improves the delivery of the drug contained therein by delivering precise, metered doses directly to the application site on the patient's body.

As used herein, the term “compounded medication” refers to a drug that is specifically prepared for a patient, based on a prescription from their doctor. The pharmacist will mix different ingredients together to create an individualized medication for the patient, in a specific strength and dosage form that is in a semi-solid composition for topical or transdermal routes of administration.

As used herein, the term “pre-determined volumetric mark” and “total dose volume” refers to the dosing knob mark that the user stops at when it is aligned with the reset arrow. In an embodiment, it is the final dose amount to be administered after setting the reset arrow at zero milliliters above the dosing knob; and each complete rotation of the dosing knob adds 1.0 ml to the total dose. In most embodiments, the user will compute the stop point before starting the dispensing, i.e. “predetermined” mark to stop at on the dosing knob.

As used herein, the term “actuations” refers to the movement of the dosing knob from one volumetric incremental mark to the next one, e.g. 0.0 ml to 0.8 ml is 8 actuations and therefore dispensing a total dose of 0.8 ml onto the dispensing cap. The number of actuations to dispense the total dose will be different if the volumetric increments are larger (e.g. 0.2 ml, 0.4 ml, etc.), so a total dose of 1.6 ml dispensed in 0.2 ml per actuation requires 8 actuations to deliver the total dose.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus, the phrase “consisting of” indicates that the listed elements are required or mandatory and that no other elements may be present.

### Drug Delivery Device

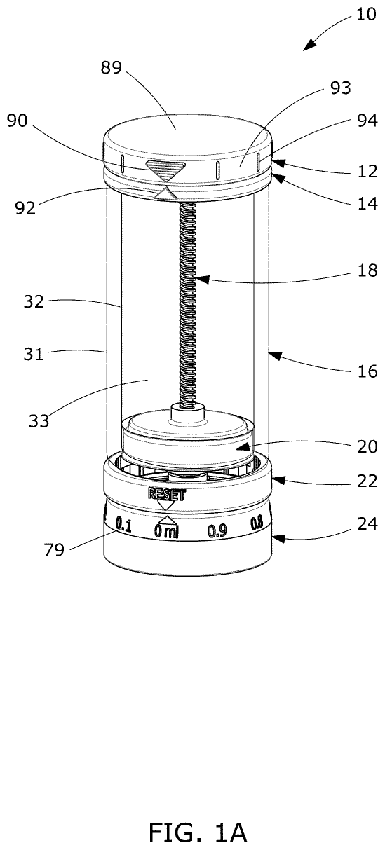

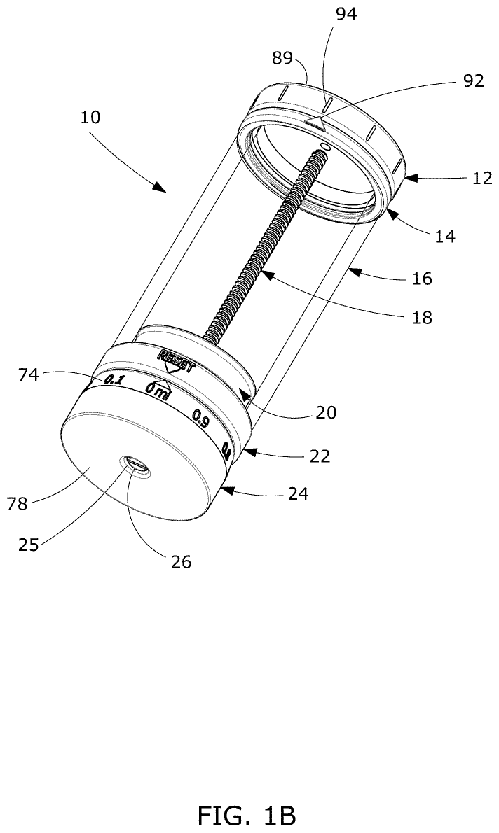

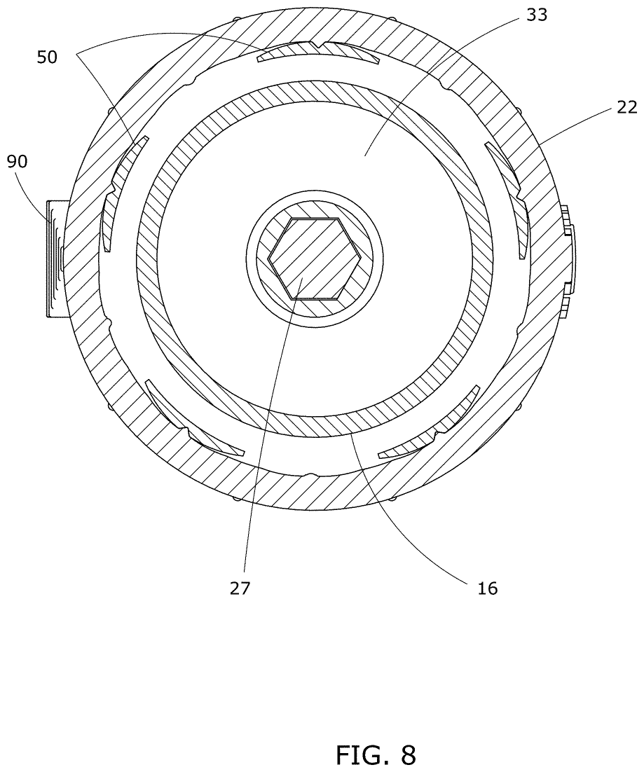

FIGS. 1A-11 illustrate an exemplary embodiment of the drug delivery device 10 for dispensing a semi-solid emulsion drug directly onto a patient's skin for topical or transdermal applications of a drug. The main components of drug delivery device 10 comprise from top to bottom: overcap 12; dispensing cap 14 with at least one hole-aperture 13; barrel 16 that holds the drug; a spindle unit 40 comprising a threaded spindle 18 connected to an elevator platform 20 that only moves upward through the barrel 16 to push the drug out of hole(s) 13; a Reset dial 22 that is not connected to the platform 20 to set before the dose is administered; and a dosing knob 24 that is connected to the platform 20, and possesses outer markings for sequential increments of drug dispensing (e.g. 0.1 milliliter per each actuation).

In an embodiment, device 10 is about 4 inches in height, and 1.5 inches in diameter; and is made of disposable materials, such as by way of non-limiting examples: one or more types of polymers, or co-polymers; one or more plastics (e.g. polypropylene), or other moldable materials used in similar devices, in which the materials are safe for contact with drugs (e.g. no transferrable contaminates, polymer leeching, etc.). The material may also be transparent or translucent to see the level of the drug remaining; and may in an embodiment comprise entirely recyclable materials.

### Overcap and Dispensing Cap

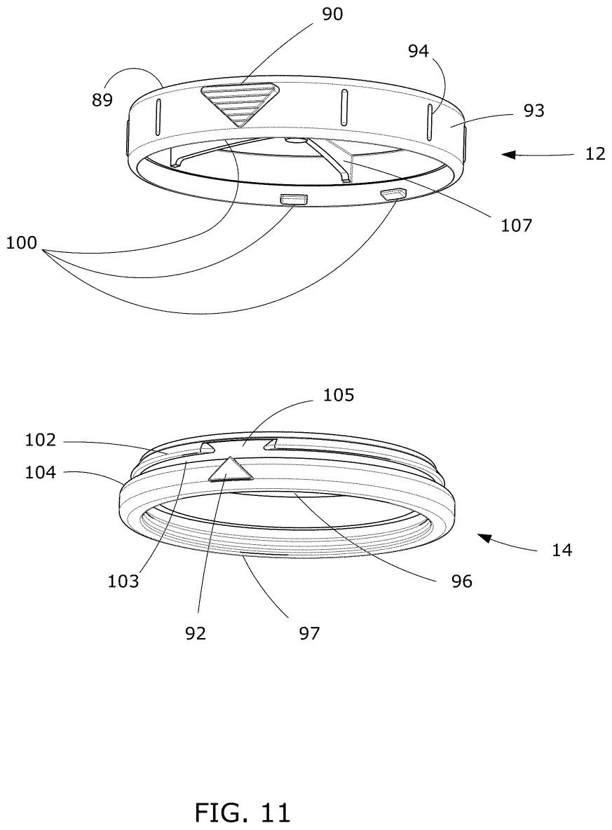

As illustrated in FIG. 11, the outer surface of overcap 12 comprises a flat top surface 89, with arrow 90 and a plurality of vertical protrusions 94 on the side wall 93 to facilitate a user snapping or rotating overcap 12 on/off dispensing cap 14 of device 10. The overcap inner surface comprises a 5-prong radial spine 107, with one tab 100 behind arrow 90 and two tabs 100 opposite arrow 90 for snapping overcap back onto dispensing cap 14 on device 10.

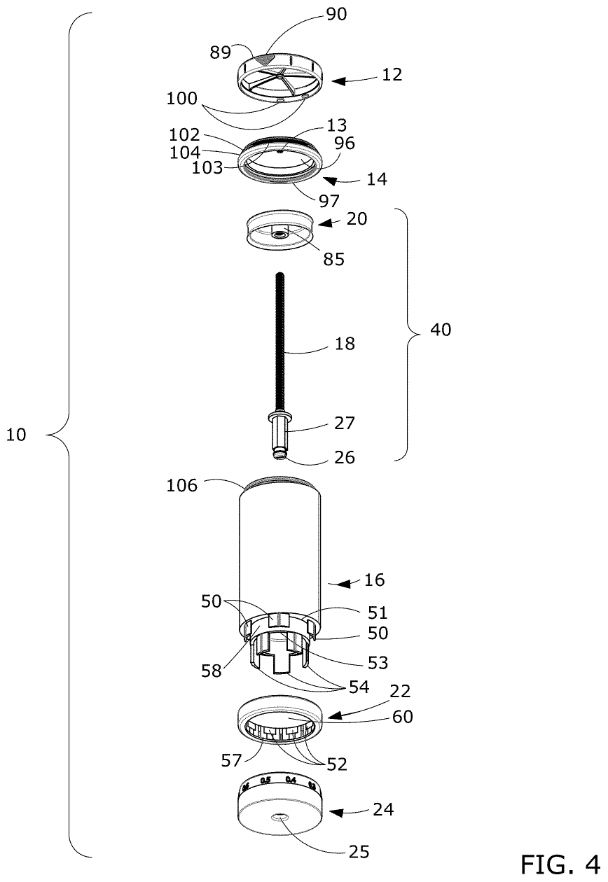

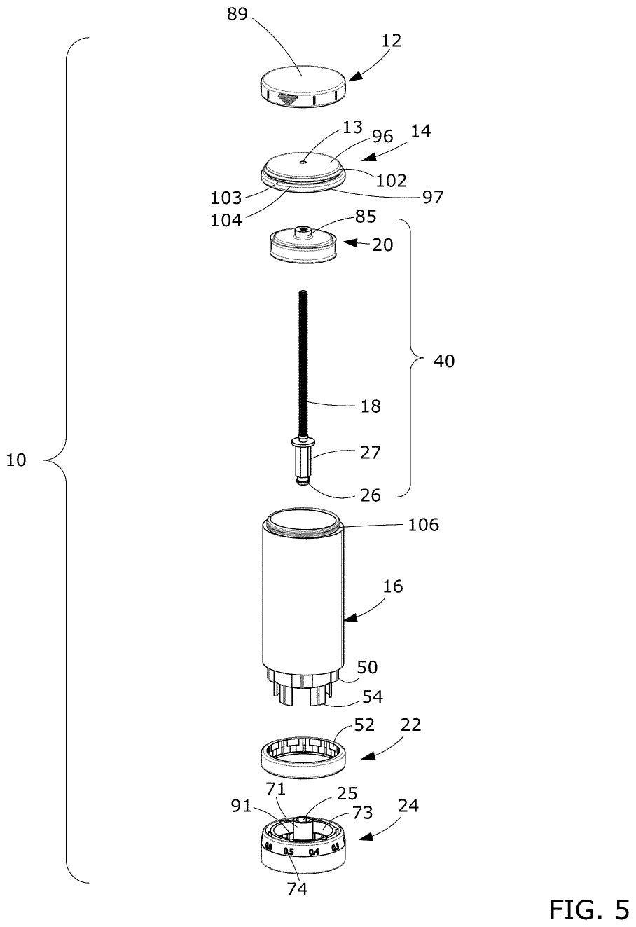

As illustrated in the exploded views of FIGS. 4-5, dispensing cap 14 comprises an outer dome shaped convex top side 96 with at least one hole or aperture 13 to dispense the drug from barrel 16 directly onto a patient's skin without contaminating the drug, while delivering a precise dose (i.e. no loss due to residue left on a user's hands, or pathogen transfer from user hands to the treatment site). In an alternative embodiment, top side 96 is flat. The width of hole(s) 13 is sufficient to easily release semi-solid compositions, no matter the viscosity, such as about 2 to 4 milliliters in diameter. The dome shape facilitates delivering the drug directly to joints, such as underarms and posterior knees.

Dispensing cap 14 further comprises: an outer wall comprising a top ring-sidewall 102 with opening 105; a bottom ring-sidewall 104 with an upward pointing arrow 92 under opening 105; and a depression ring 103 positioned under ring 102 for attaching the three tabs 100 on overcap 12.

Childproof cap: as illustrated in FIG. 1A, and 11, overcap 12 and dispensing cap 14 are childproof by preventing a child from easily snapping the overcap off the device 10. Overcap 12 downward pointing arrow 90 has a textured surface to facilitate a user pushing upward on it to snap the overcap off. A user must first rotate overcap 12 until overcap arrow 90 aligns with dispensing arrow 92 and opening 105 in order to snap overcap 12 off device 10.

When the user needs to reattach the overcap 12 onto the device 10 again after administering the drug, the user does not need to line up dispensing cap's tab 92 with the overcap's tab 90. Instead, the user fits the two overcap tabs 100, which are on the overcap inner wall opposite arrow 90, into a depression ring 103 under ring 102 on the dispensing cap sidewall.

Dispensing cap 14 may have a color indicating a particular type of drug that it dispenses, which is especially useful for patients and/or pharmacists using more than one device 10 in order to avoid confusion and accidental administration of the incorrect drug. The entire dispensing cap 14 is color coded.

In an embodiment, device 10 and caps 12 and 14 have a circular shape along the horizontal cross-sectional area.

### Barrel, Reset Dial, Dosing Knob

As illustrated in FIGS. 1A, 2-5, 7A, and 7B, barrel 16 comprises a hollow tubular member comprising an inner wall 32 and an outer wall 34, a top end 36 and a bottom end/side 51. Circular inner wall 32 forms a cavity 33 that is a longitudinally oriented cylinder (e.g. tubular shaped), with an elliptical cross-sectional area able to house a plurality of doses of semi-solid composition (e.g. drug) without leakage from the barrel. In an exemplary embodiment, cavity 33 stores a prescription of a drug from Table 1, or any combination thereof, which comprises about 1.4 ounces.

Centered within barrel 16 is the spindle unit 40, comprising from top to bottom as shown in the exploded views of FIGS. 4, 5, 10A, and 10B: 1) elevator platform 20; 2) threaded screw 18; and 3) connection unit 27. Elevator platform 20 is horizontally oriented (i.e. extending across the cross-sectional area of barrel 16 to form a fluid seal) and comprises in an embodiment: a thin flat elliptical disc top side 81; a center threaded hole 84 in a vertical tube 85 that fits screw 18; and an elliptical flat sidewall 82.

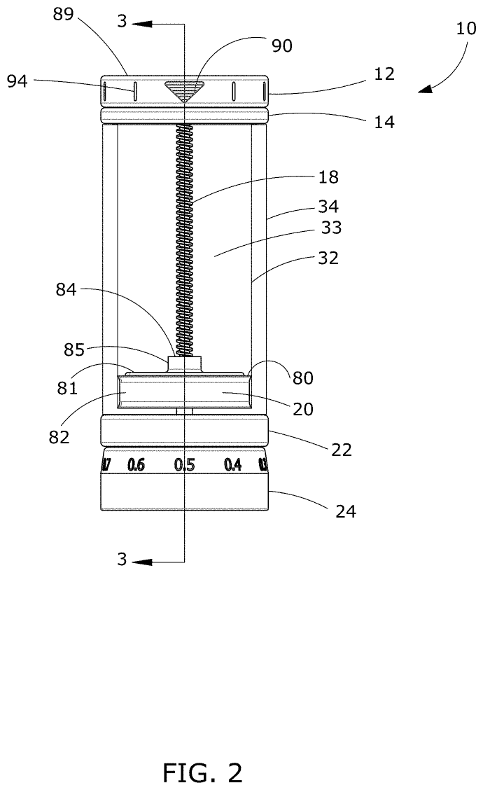

As illustrated in FIGS. 2, 3 and 10B, platform 20 may further comprise an elliptical shaped channel 80 on the disc 81 outer periphery that functions as a drug reservoir or canal for storing drug backflow as the platform rises in order to form a fluid seal between barrel inner wall 32 and platform 20. The bottom of sidewall 82 of platform 20 may further comprise a downward and outward pointed ring 86 to further provide a fluid seal. Thus, platform sidewall 82 in a side view comprises a vertical wall with an outward flared top and bottom edge.

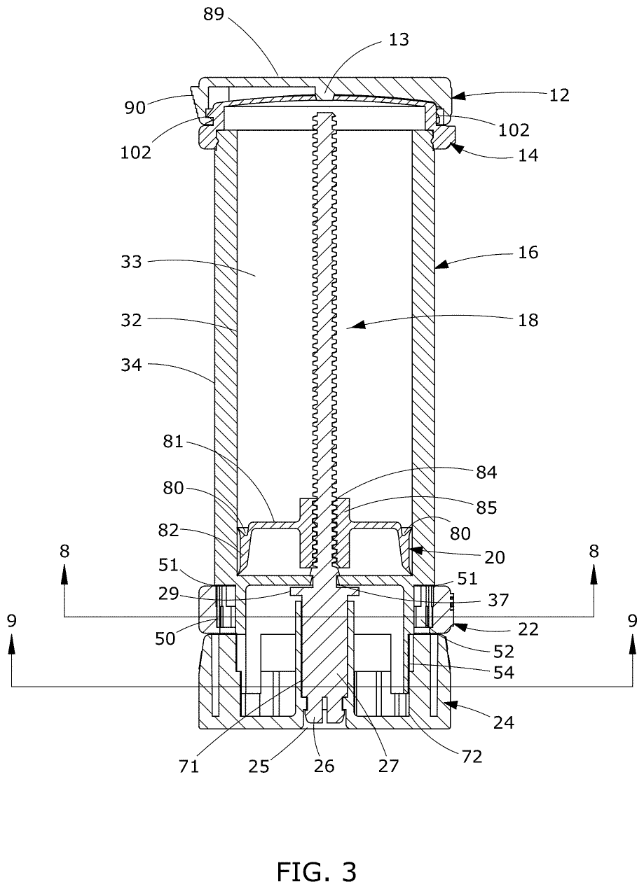

As illustrated in FIGS. 3-5, platform 20 may comprise a variety of horizontal cross-sectional shapes (e.g. circular, oval, elliptical, etc.), as long as it covers the entire horizontal cross-sectional area of barrel 16 to form a liquid seal that blocks seepage of the semi-solid composition below platform 20. For example, if barrel 16 cavity has an elliptical cross-sectional area, then platform 20 has the same elliptical shape and is sized to slide up the barrel inner wall 32 without rotating while preventing any seepage of the drug beneath platform 20. Drug seepage is also deterred by top channel 80 on the outer circumference of the platform top side 81, which pushes the drug away from the barrel inner wall 32 and into the ring 80 as platform 20 rises with each actuation of dosing knob 24.

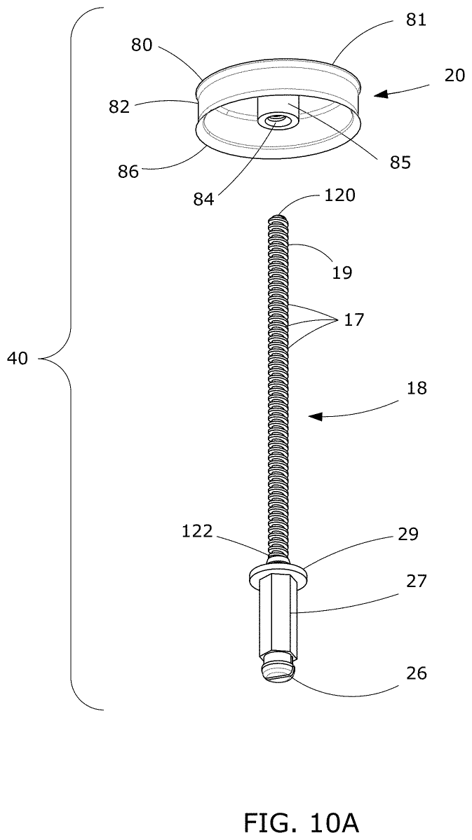

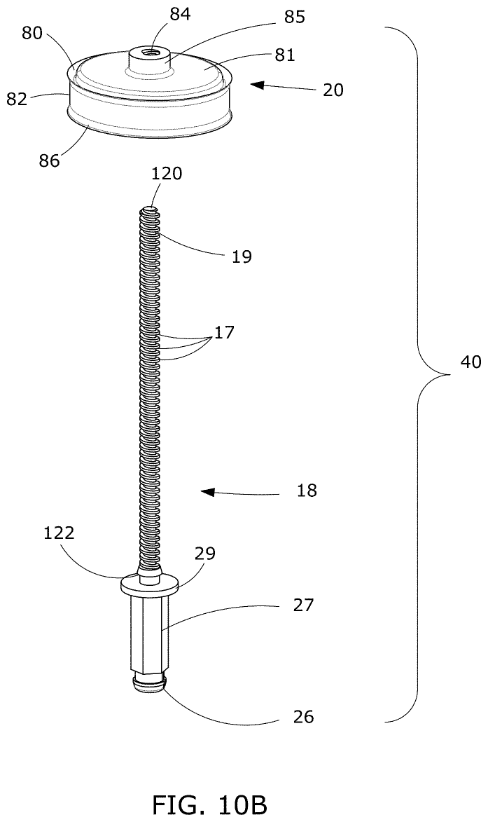

As illustrated in FIG. 10A, threaded screw 18 comprises: an elongated shaft 19 centered vertically within the middle of barrel 16; a plurality of external threads 17 upward rotating; a top end 120; and a bottom end 122; Threaded screw 18 is able to connect securely with elevator platform hole 84 to move the platform 20 upward through barrel 16 with each actuation of dosing knob 24 while screw 18 rotates in place without rising (e.g. similar to deodorant sticks).

As shown in FIGS. 3-5, and 10A, the bottom end 122 of screw 18, beneath platform 20, is jointed to a connection unit comprising from top to bottom: a washer or screw plate 29; a solid tubular member 27 without threads (e.g. octagonal, cylindrical, square shaped, etc.); and a circular end 26 (e.g. a screw head). Washer 29 rests against the underside of barrel bottom hole 37, as shown in FIG. 3, to prevent the bottom of the spindle unit 40 from being dislodged into the barrel cavity 33. The shape of unit 27 matches dosing knob's central tubular channel 71 with hole 25. For example, and as shown in FIGS. 4, 5, and 7B, if channel 71 has a hexagon shaped inner surface, then screw connection unit 27 has the same hexagon shape. Unit 27 is also long enough to extend vertically through the center of reset dial 22 and dosing knob 24 such that screw end 26 snaps into channel 71 and hole 25 in the dosing knob 24 bottom end.

The bottom end of barrel 16 further comprises an inner ring and an outer ring of protrusion members 50, 54, respectively (e.g. protrusions are male rectangular or square extensions) to control the movements of the reset dial 20 and the dosing knob 24 while maintaining the barrel in a fixed position. In an embodiment as illustrated in FIGS. 4, 5, 7A, and 7B, the bottom end 51 of barrel 16 comprises a plurality of short substantially square shaped protrusion members 50 extending vertically downward positioned in a ring near the edge of barrel end 51 and curved inward (convex). Protrusions/Tabs 50 fit into the slots 52 of reset dial 22 inner wall 57. With this connection, reset dial 22 is able to freely rotate clockwise and counterclockwise around barrel 16 without rotating the barrel or dispensing knob 24. Rotating reset dial 22 while barrel 16 and prongs 50 are held in position, forces reset dial slots 52 to move to the next barrel protrusion members 50 while eliciting a clicking noise with each actuation.

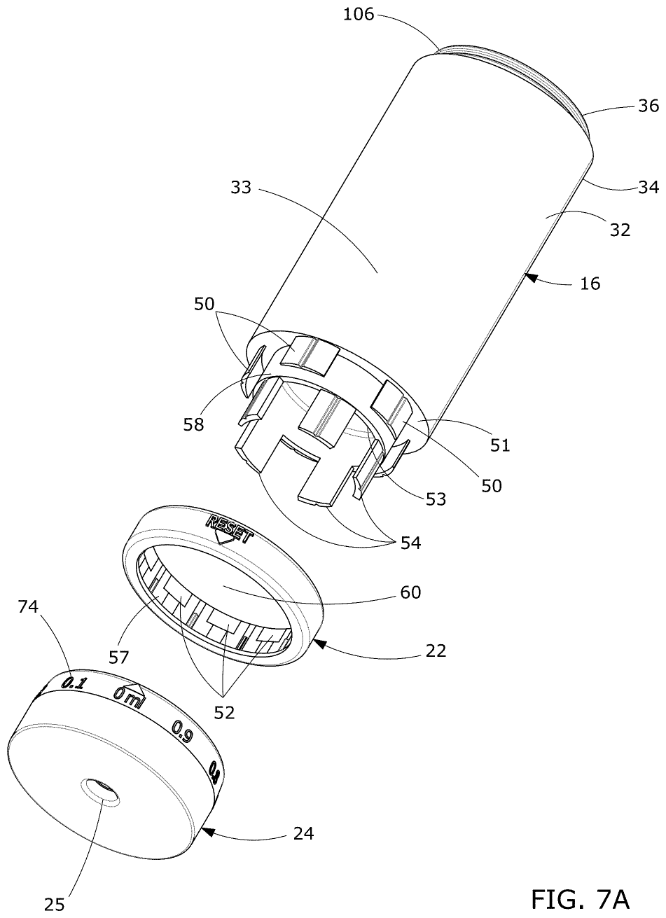

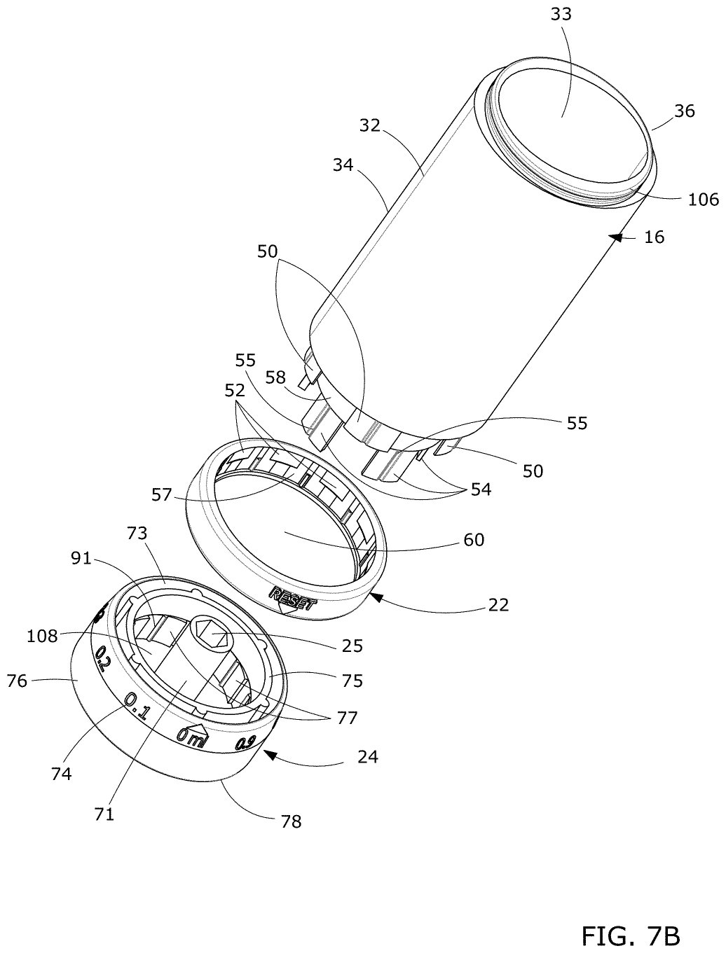

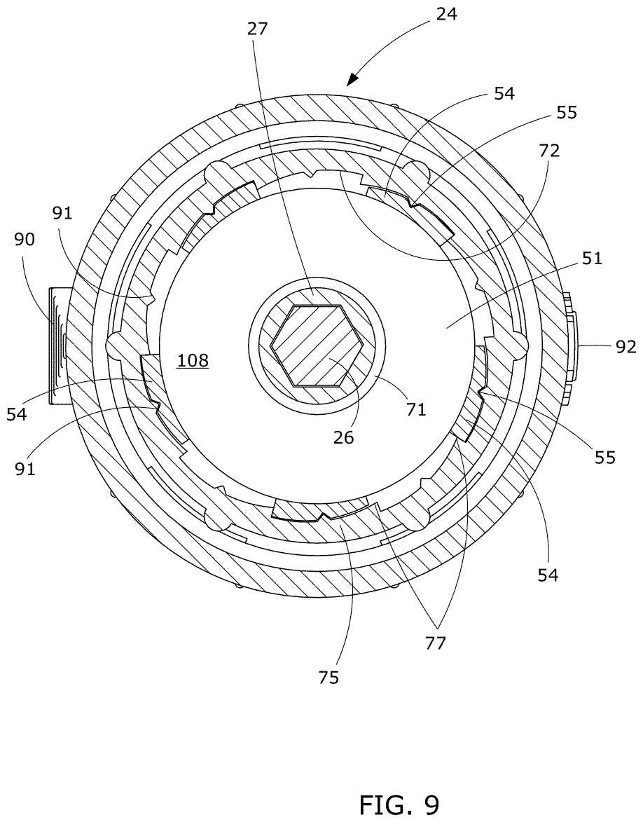

Dosing Knob 24: as illustrated in FIGS. 7B and 9, dosing knob 24 comprises: a flat circular bottom end 78 with a center hole 25 and vertical channel 71 extending up the middle of the knob. Knob 24 further comprises an outer sidewall 76 with incremental dose markings 74 spaced equal distance around the sidewall (e.g. 0 ml, 0.1, 0.2, 0.3, 0.4, 0.5, 0.6, 0.7, 0.8, 0.9). Knob 24 further comprises an inner ring 75, facing the knob inner ring wall 73. As illustrated in FIGS. 7B and 9, knob inner ring wall 73 on the bottom comprises 10 equidistant spaced tubular extensions 91 that fit into barrel vertical grooves 55 on barrel protrusions 54. Hence, barrel protrusions 54 extend through the reset dial cavity 60, and the dosing knob inner cavity 108 to connect with the knob inner wall tubular extensions 91. Aligned next to tubular extensions 91 are also vertical ridges 77 to block dosing knob's accidentally slippage via clockwise and counterclockwise rotation.

The connections between parts 91, 77, and 54 can create an audible clicking noise and/or a physical sensation in the user's hand with each actuation of dosing knob 24 to facilitate allowing the user to know that they are correctly rotating the dosing knob. The connections also prevent dosing knob 24 from rotating clockwise accidentally, and from slipping backward in a counterclockwise fashion; thus, ensuring that a precise unit of dose is released from dispensing cap 14 per each actuation. For example, tubular extensions 91 fit into grooves 55 on flexible barrel protrusions 54 to stabilize dosing knob 24 and to provide the perfect amount of resistance for the user to rotate dosing knob 24. When the user actuates dosing knob 24, protrusions 91 rotate to the next vertical groove 55. This connection (91/55) prevents accidental actuation of dosing knob 24, e.g. it prevents situations like where the patient stores the device in her handbag and it accidentally actuates due to inadvertent movement of the bag. An actuation of the device must be intentional, and the resistance provided by the connection between components 91 and 55 provides that.

Additionally, dosing knob vertical ridges 77 protrude into inner cavity 108 just like vertical extensions 91, but in a gradual ramp-like manner so that barrel protrusions 54 can still rotate when intentionally manually turned. Yet once the device has been actuated clockwise (as viewed from the device bottom), dosing knob ridges 77 block protrusions 54 from slipping backwards or counterclockwise because protrusions 54 cannot slip over ridges 77 (i.e. barrel 54's edges fit up against dosing knob ridges 77). This therefore prevents the dosing knob 24 from slippage via rotating counterclockwise.

Thus, by intentionally manually turning dosing knob 24 clockwise (as viewed from the bottom of device 10) one or more actuations, then threaded screw 18 simultaneously rotates clockwise without slippage due to the rotation of knob 24's channel 71, and platform 20 moves vertically up screw 18 while pushing a precise dose of the semi-solid composition out of hole(s) 13.

### Method of Assembly

In an embodiment, device 10 is assembled in the following steps. Elevator platform 20 is inserted into an empty barrel 16 and pushed down to the barrel bottom. Reset dial 22 is snapped onto the barrel bottom end 51 by fitting into protrusions or prongs 50 that extend from the barrel bottom in an outer ring (e.g. see FIG. 7A, 7B). As illustrated in FIGS. 3-5, 10A, and 10B, threaded spindle 18 is inserted through the center of reset dial 22, and is then screwed or pushed through opening 37 in the barrel bottom 51 up to the spindle's washer 29. Spindle 18 is then screwed through the center hole 84 of platform 20. Dosing knob 24 is then snapped onto barrel 16, beneath reset dial 22, by fitting into barrel protrusions 54 that extend from the barrel bottom inner ring and also by aligning channel 71 to attach to connection unit 27. The device 10 is then filled with a specific volume of the prescription drug into barrel 16. Dispensing cap 14 is then snapped onto barrel 16. Overcap 12 is then snapped onto dispensing cap 14.

### Reset Dial and Dosing Knob, Methods of Use

Device 10 is a disposable drug delivery system (i.e. filled with a drug). For example, a pharmacy fills the device with a patient's prescription, which may last weeks, months, or years, depending on the drug. The pharmacist fills device 10 by snapping overcap 12 and dispensing cap 14 off device 10, and pushing platform 20 down to the bottom of barrel 16. The pharmacist then fills barrel 16 with the prescribed drug or a compounded medication without contaminating it using filling procedures well known in the pharmacy industry. As illustrated in FIGS. 5 and 11, the pharmacist then snaps dispensing cap 14 onto barrel 16, and snaps overcap 12 onto the dispensing cap 14 using overcap's tabs 100 that attach to ring 102 on dispensing cap's outer wall.

The drug delivery device should dispense all of the drug as intended by the prescription when and if the patient is administering the amount and frequency of the dose as prescribed. The patient then discards device 10 in the garbage; and obtains another prescription and device if required.

The user has various methods available to compute and/or monitor how many actuations are required to administer their dose.

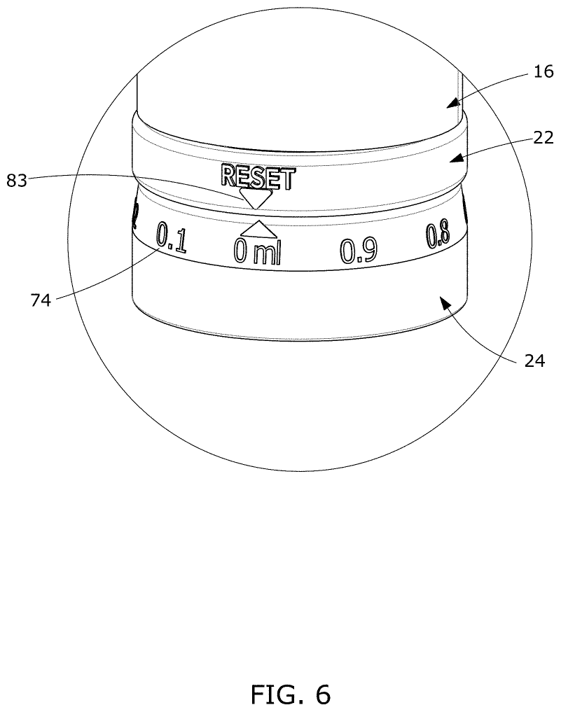

FIG. 6 illustrates a close-up view of reset dial 22 and dosing knob 24. Reset dial 22 further comprises markings 83 on the dial (e.g. “RESET” and an arrow pointing downward towards dosing knob's total dose amount 74).

To administer the drug, first the user determines how many mini doses or units (e.g. actuations) of device 10 are required to administer the full dose. This requires the user to divide the total dose in milliliters by 0.1 milliliters per actuation to compute the total number of actuations required. It is noted that other devices of the present invention may have different increments 74 on dosing knob 24 (e.g. 0.2 ml increments, 1 ml, etc.), based on the drug class and condition or disease being treated.

Then in the preferred embodiment, every time the user is about to administer a dose of the drug, they rotate the “Reset” arrow 83 to stop above “0 ml” on knob 24 first. They then administer the drug in incremental amounts, as indicated on knob 24. For example, each actuation of knob 24 will dispense 0.1 milliliters of the drug directly onto a user's skin. If the total dose is 0.2 milliliters, then the user stops when the reset dial arrow 83 is aligned with the dosing knob 74 text at 0.2 milliliters. Likewise, if the total dose is two milliliters, the user stops dispensing when dosing knob 24 rotates two full rotations around the device and stops at “0 ml” below Reset arrow 83, a second time. And if the total dose is 1.4 milliliters, the user moves the Reset arrow 83 to “0 ml”, then performs 14 actuations, which they do not need to mentally count. Instead, they will know when to stop when the dosing knob rotates one time around device 10 and stops at “0.4 ml” below reset arrow 83.

In another embodiment, the user does not need to move the Reset arrow 83 to the 0 ml mark 74. They just need to mentally count the numbers of actuations of knob 24 are needed to administer an entire dose; and/or they just need to know where the stop point 83 is. For example, if a user needs to dispense a 0.8 ml dose, then they need to count out 8 actuations of knob 24 or determine before dispensing where knob 24 stops (e.g. adding start point of 0.2 ml+0.8 ml, and stop point is 0 ml aligned with reset arrow 83).

The dose is applied topically or transdermally to skin areas as prescribed. For example, when the drug is for hormone replacement therapy, then the dose is administered to the patients transdermally at one or more of: inner/under arms; inner and back thighs; stomach; back; etc. Device 10 and hormone drugs are not intended for internal use, e.g. mouth, nose, eye, vaginal, etc.

There are also various methods of use of the device of the present invention. In all embodiments, the method comprises: manually, and incrementally, turning the dosing knob in one hand while holding the barrel still in the opposing hand, wherein each actuation of the dosing knob dispenses one unit of the semi-solid composition dose from the dispensing cap hole(s) by pushing the elevator platform up the threaded spindle; and b) administering the entire dose at once (e.g. by allowing it to collect on top of the dispensing cap), or each dose unit one-at-a-time, in a precise amount of the semi-solid composition from the dispensing cap directly onto a user's skin.

In another embodiment, the method of use further comprises: a) aligning a reset dial arrow with a zero-milliliter setting on the dosing knob before activating the dosing knob; b) manually turning the dosing knob to a pre-determined volumetric mark (i.e. the final dose), one actuation at a time, until the mark aligns with the reset dial arrow; and c) wherein the pre-determined volumetric mark comprises a dose total volume to be dispensed, and the user is not required to keep count of the number of actuations to determine when the dose is fully administered. Therefore, the user just keeps turning the dosing knob until the final dose is aligned with the reset arrow.

If the dose is more than 1 ml (e.g. 1.4 ml), the user subtracts 1 ml from the dose total volume, and moves the dosing knob to 0.4 ml to administer the total dose. The user will need to pay attention that they have rotated the dosing knob at least one full 360 degrees, before stopping the dosing knob at 0.4 ml (i.e. 14 actuations). If the dose is more than 1 ml, then the user must first turn the reset dial to the 0 ml volumetric mark to reset the device, then rotate the dosing knob 360 degrees (or a full rotation) the number of times that the dose calls for in whole numbers, then dial the remaining dose to the pre-determined volumetric mark. So for example if the dose is 2.4 ml, the whole number is 2, so the user will reset the device to 0 ml, then rotate the dosing knob 2 full turns, then the remainder is 0.4 ml, so then the user will rotate the dosing knob to the 0.4 ml mark, which is 24 actuations.

In another embodiment, the method of use further comprises: a) computing by the user the final dosing knob stopping mark by summing a pre-determined volumetric mark (i.e. total dose) with a current volumetric mark (i.e. where is reset arrow pointing at start); and b) manually turning the dosing knob to the final mark, one actuation at a time; and c) wherein the user is not required to keep count of the number of actuations to determine when the dose is fully administered. In this version, the user does not have to keep moving the reset dial to zero. They just need to take into account the starting point of the reset dial.

If the user forgets to note the reset dial starting point, all is not lost. Instead the user can just count actuations, the method comprising: a) computing by a user a total number of actuations comprising a dose total volume divided by a volume dispensed per each knob actuation (e.g. 0.8 ml dose total volume/0.1 ml dispensed per actuation=8 actuations); and b) manually turning while counting the dosing knob one actuation at a time, until the total number of actuations is completed. If the user does not first mark the starting point (e.g. moving reset dial to 0 ml start), then the user must count the number of actuations and stop at 8.

### Drug Delivery System

The present invention further comprises a drug delivery system comprising device 10 filled with a topical or transdermal drug. Topical drugs are well known in the art that can be administered directly to a patient's skin at the site of affliction, such as by way of non-limiting examples: to treat a localized skin infection (e.g. cellulitis, ringworm, acne, etc.), inflammation (e.g. blisters, swelling, redness, etc.), insect bite, or a chronic condition (psoriasis, rosacea, dermatitis, etc.).

In another embodiment, the drug delivery system comprises device 10 filled with a transdermal drug that passes through the skin when applied topically, to enter the patient's circulatory system. With either topical or transdermal drugs, device 10 is applied directly to the skin, without contacting a user's hands, in order to prevent contamination of the dispensing cap 14, and to deliver precise amounts of drug doses.

In an exemplified embodiment, the transdermal drug is for hormone replacement therapy and/or a compounded medication to treat or prevent a medical condition or disease. Transdermal bio-identical hormone replacement cream is well known in the art and is absorbed through skin into the systemic circulation to replace endogenous hormones. The drug is biologically identical to human produced hormones.

A non-limiting list of exemplary types of transdermal drugs for use in hormone replacement therapy is listed below in Table 1. The drugs are divided into the general categories of: progestogen; estrogen; androgen; thyroid; and others.

TABLE 1

| Drug Category | Drug Generic Name |
|---|---|
| Estrogen | Estradiol and all pro-drug esters thereof, estradiol |
| | acetate, estradiol benzoate, estradiol cypionate, |
| | estradiol dienanthate, estradiol valerate, ethinyl |
| | estradiol, estradiol succinate |
| Progestogen | Progesterone and all pro-drug esters thereof |
| | Dydrogesterone, hydroxyprogesterone, |
| | hydroxyprogesterone caproate, medroxyprogesterone |
| | acetate, megestrol acetate, 17-hydroxy-progesterone |
| Androgens | Testosterone and all pro-drug esters thereof, |
| | testosterone cypionate, testosterone enanthate, |
| | testosterone propionate, testosterone undecanoate. |
| Thyroid | Levothyroxine sodium pentahydrate, liothyronine |
| Others | Dehydroepiandrosterone (DHEA) and all pro-drug |
| | esters thereof, DHEA sulfate, 7-keto-DHEA, DHEA |
| | acetate |
| | Pregnenolone and all pro-drug esters thereof, |
| | pregenenolone acetate, pregnenolone sulfate sodium |
| | Human chorionic gonadotropin (HCG), somatotropin, |
| | vasopressin, oxytocin, aldosterone |
| Non-Steroidal | Diclofenac, ibuprofen, ketoprofen, piroxicam, and all |
| Anti- | other NSAIDs |
| Inflammatory | |
| Drugs (NSAIDs) | |
| Steroidal | Betamethasone, hydrocortisone, dexamethasone, |
| anti-inflammatory | clobetasol, and all steroidal anti-inflammatory drugs |
| drug | |
| Analgesics | Ketamine, lidocaine, tramadol |
| Muscle relaxants | Cyclobenzaprine, baclofen, carisoprodol |
| Nerve modulating | Amitriptyline, gabapentin, pregabalin, clonidine |
| agents | |

Dehydroepiandrosterone (DHEA), (chemical formula C₁₉H₂₉O₂; and molar mass 288.424 g/mol) is also known as androstenolone, which is an endogenous steroid hormone produced in the adrenal glands, the gonads, and the brain. DHEA is converted into male and female sex hormones, such as estrogen and testosterone, and is often prescribed to patients to protect against health problems associated with hormone imbalances and/or aging-related declines in hormone levels, such as: Alzheimer's prevention, reduction; Parkinson's; chronic fatigue syndrome; depression; erectile dysfunction; fatigue; fibromyalgia; lupus, menopausal symptoms, metabolic symptoms and syndrome; multiple sclerosis; improve sports performance by increasing muscle and reducing fat, enhance libido, promote weight loss, and bolster the immune system.

Estradiol (chemical formula C₁₈H₂₄O₂ and a molecular mass of 272.38 g/mol) is an estrogen steroid hormone that is used to treat menopause symptoms such as hot flashes and vaginal changes, and to prevent osteoporosis (bone loss) in menopausal women. Estradiol is also used to treat low estrogen levels in women with ovarian failure. It is also indicated to treat certain types of breast cancer and prostate cancer.

Progesterone (C₂₁H₃₀O₂ and a molar mass 314.469 g/mol) is a naturally occurring steroid hormone. It is used in combination with estrogens mainly in hormone therapy for menopausal symptoms, for low sex hormone levels in women, to support pregnancy and fertility, and to treat gynecological disorders.

Testosterone (C₁₉H₂₈O₂ and a molar mass: 288.42 g/mol) is the primary male sex hormone and anabolic steroid. In male humans, testosterone plays a key role in the development of male reproductive tissues such as testes and prostate, as well as promoting secondary sexual characteristics such as increased muscle and bone mass, and the growth of body hair. A topical gel of the present invention may be used to treat, in an embodiment, males whose bodies do not make enough natural testosterone, a condition called hypogonadism.

The daily dosage for the various drugs described herein can be administered to a patient in a single dose (e.g. multiple actuations of device 10 in one setting). A patient may require more than one dose per day; and/or the dose may change from day-to-day, or per the time of day.

The drugs of the present invention can further comprise one or more pharmaceutically acceptable carriers, one or more excipients and/or one or more additives. The pharmaceutical compositions can comprise about 1 to about 99 weight percent of active ingredient (e.g. Table 1), and preferably about 5 to about 30 percent active ingredient.

Useful pharmaceutically acceptable carriers are semi-solid emulsions comprising, by way of non-limiting examples: creams, ointments, pastes, gels. The amount of carrier in the drug can range from about 5 to about 99 weight percent of the total weight of the drug.

Non-limiting examples of suitable pharmaceutically acceptable excipients and additives include moisturizers, surfactants, solvents, acids, oils, perfumes, antiseptics, preservatives, anti-oxidants, lubricants, thickeners, humectants, salts, disinfectants, binders, film forming agents, skin softening agents, coloring agents, wetting agents, emulsifiers and the like. The amount of excipient or additive can range from about 0.1 to about 95 weight percent of the total weight of the drug. One skilled in the art would understand that the amount of carrier(s), excipients and additives (if present) can vary by the drug formulation and the disease or condition being treated.

### CONCLUSION

A phrase such as “an aspect” or “object” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as an “aspect” may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as a “configuration” may refer to one or more configurations and vice versa.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology.

The exemplary device, system, and methods described in the embodiments presented previously are illustrative, and, in alternative embodiments, certain acts can be performed in a different order, in parallel with one another, omitted entirely, and/or combined between different example embodiments, and/or certain additional acts can be performed, without departing from the scope and spirit of various embodiments. Accordingly, such alternative embodiments are included in the inventions described herein.

Although specific embodiments have been described above in detail, the description is merely for purposes of illustration. It should be appreciated, therefore, that many aspects described above are not intended as required or essential elements unless explicitly stated otherwise. Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

The product names used in this document are for identification purposes only. All trademarks and registered trademarks are the property of their respective owners.

What is claimed is:

1. A disposable, drug delivery device 10 for metered dosing of semi-solid compositions, comprising:
a hollow barrel 16 with a tubular outer wall 34 and inner wall 32 forming a cavity 33 within, wherein the barrel is able to store a plurality of doses of a semi-solid composition within the cavity, and without leakage of the composition from the device;
a threaded spindle unit 40, centered vertically within said barrel, comprising: 1) an elevator platform 20 comprising a thin, horizontal, flat disc top side 81 with a centered threaded hole 84, and 2) a threaded screw 18 passing through the threaded hole;
a reset dial 22 displaying a reset downward pointing arrow 83, the dial positioned below the barrel, and able to manually rotate bi-directionally without turning the barrel, the spindle unit, or a dosing knob;
the dosing knob 24 connected beneath the reset dial, and able to be manually and incrementally rotated unidirectionally per each knob actuation;
a dispensing cap 14 connected to a top of said barrel, comprising at least one centered hole 13 wide enough to release a unit of the composition;
an overcap 12 covering the dispensing cap when the device is not in use, able to prevent contamination of the dispensing cap and evaporation of the composition;
wherein the reset dial is position-able above and aligned with a user's pre-determined setting on the dosing knob to enable a user to dispense a dose of the semi-solid composition comprising a plurality of units and without having to count the actuations; and
wherein each manual actuation of the dosing knob dispenses a unit of the semi-solid composition dose from the dispensing cap hole(s) by incrementally pushing the elevator platform up the threaded spindle.

2. The disposable, drug delivery device of claim 1, wherein the elevator platform 20 and the cavity 33 inside the barrel 16 further comprise an elliptical shaped horizontal cross-sectional area.

3. The disposable, drug delivery device of claim 1, wherein the dosing knob displays a plurality of equidistance sequentially numbered volumetric markings 74 around the entire circumference of a knob outer wall 76.

4. The disposable, drug delivery device of claim 3, wherein the volumetric markings comprise ten markings counting from 0 milliliters to 0.9 milliliters.

5. The disposable, drug delivery device of claim 3, wherein the reset arrow is able to be manually, and bi-directionally aligned with the dosing knob volumetric markings while holding the barrel in position, while emitting an audible click with each reset dial actuation.

6. The disposable, drug delivery device of claim 1, further comprising the dispensing cap being colored to indicate the semi-solid composition that is stored within the barrel.

7. The disposable, drug delivery device of claim 1, wherein the overcap is childproof comprising a textured arrow 90 on overcap sidewall 93 and an inner tab 100 behind textured arrow 90 that is align-able with an opening 105 and an arrow 92 on the dispensing cap sidewall 104 in order for an adult to remove the overcap from the device.

8. The disposable, drug delivery device of claim 7, wherein the overcap further comprises two inner tabs positioned opposite said textured arrow 90, and the dispensing cap sidewall 104 further comprises a middle depression ring 103 to enable snapping the overcap onto the dispensing cap, without aligning arrows 90 and 92, by fitting the two inner tabs 100 into the depression ring 103.

9. The disposable, drug delivery device of claim 1, wherein the elevator platform further comprises: a vertical tube 85 housing the center threaded hole 84 in the center of the platform top side; and an elliptical side wall 82.

10. The disposable, drug delivery device of claim 9, wherein the elevator platform further comprises: an elliptical channel 80 on the top side 81 periphery, which is able to hold a drug backflow to form a liquid seal that prevents a drug leakage from the barrel.

11. The disposable, drug delivery device of claim 1, wherein the barrel further comprises a flat bottom end 51 with two concentric rings of protrusion members 50 outer, and 54 inner, extending vertically downward, said protrusions able to connect the barrel to the reset dial and the dosing knob, respectively.

12. The disposable, drug delivery device of claim 10, wherein the reset dial further comprises an inner wall 57 with a plurality of slots 52, wherein the protrusion members 50 fit into said slots enabling the reset dial to rotate bi-directional around the barrel and the dosing knob.

13. The disposable, drug delivery device of claim 10, wherein the barrel inner protrusion members 54 extend from the barrel bottom end 51 through a reset dial circular cavity 60 and a dosing knob cavity 108 to connect without slippage to 5 of the 10 equidistant vertical grooves 91 on a dosing knob inner wall 73 to release a precise dose of the drug per each knob actuation.

14. The disposable, drug delivery device of claim 12, wherein the overcap is childproof comprising an outer textured arrow 90 on overcap sidewall 93 and an inner tab 100 behind arrow 90 that is align-able with an opening 105 and an arrow 92 on the dispensing cap sidewall 104 in order for an adult to remove the overcap from the device.

15. The disposable, drug delivery device of claim 1, wherein:
the threaded spindle unit further comprises a connection unit with a solid tubular member 27 without threads and a circular end 26 positioned at the bottom end of screw 18, beneath the elevator platform 20;
the dosing knob further comprises a centered hollow channel 71 with a bottom hole 25; and
the solid tubular member 27 and the circular end 26 fit into the centered hollow channel 71 and the bottom hole 25 to connect the threaded spindle unit to the dosing knob.

16. The disposable, drug delivery device as in one of claim 1, further comprising a system having stored within the barrel and above the elevator platform, one or more doses of a semi-solid composition using one or a combination of active drug ingredients as shown in Table 1.

17. A method of dispensing a semi-solid composition with metered dosing from a disposable, drug delivery system, comprising:
a) providing a disposable drug delivery system, comprising:
a hollow barrel 16 with a tubular outer wall 34 and inner wall 32 forming a cavity 33 within, wherein the barrel is able to store a plurality of doses of a semi-solid composition within the cavity, and without leakage of the composition from the device;
a threaded spindle unit 40, centered vertically within said barrel, comprising: 1) an elevator platform 20 comprising a thin, horizontal, flat disc top side 81 with a centered threaded hole 84, and 2) a threaded screw 18 passing through the threaded hole;
a reset dial 22 displaying a reset downward pointing arrow 83, the dial positioned below the barrel, and able to manually rotate bi-directionally without turning the barrel, the spindle unit, or a dosing knob;
the dosing knob 24 connected beneath the reset dial, and able to be manually and incrementally rotated clockwise per each knob actuation;
a dispensing cap 14 connected to a top of said barrel, comprising at least one centered hole 13 wide enough to release a unit of the composition;
an overcap 12 covering the dispensing cap when the device is not in use, able to prevent contamination of the dispensing cap and evaporation of the composition;
b) aligning a reset dial arrow with a dosing knob zero-milliliter volumetric mark;
c) manually, and incrementally, turning the dosing knob clockwise while holding the barrel in position, wherein each actuation of the dosing knob dispenses one unit of the semi-solid composition dose from the dispensing cap hole(s) by pushing the elevator platform up the threaded spindle;
d) stopping the dosing knob when a pre-determined dose volumetric mark is aligned with the reset arrow indicating an entire dose has been dispensed, wherein the user is not required to keep count of the number of dosing knob actuations; and
e) administering a precise amount of the one or more dose units of the semi-solid composition from the dispensing cap directly onto a user's skin.

18. The method of dispensing a semi-solid composition of claim 17, wherein when the entire dose is greater than 1 milliliter, then the pre-determined dose volumetric mark comprises subtracting the whole number from the dose and rotating the dosing knob more than a full rotation.

19. The method of dispensing a semi-solid composition of claim 17, wherein the volume dispensed per each knob actuation is 0.1 milliliters.

20. The method of dispensing a semi-solid composition of claim 17, wherein the elevator platform 20 and the cavity 33 inside the barrel further comprise an elliptical shaped horizontal cross-sectional area.

21. The method of dispensing a semi-solid composition of claim 17, wherein the dosing knob displays a plurality of equidistance sequentially numbered volumetric markings 74 around the entire circumference of a knob outer wall 76.

22. The method of dispensing a semi-solid composition of claim 21, wherein the volumetric markings comprise ten markings counting from 0 milliliters to 0.9 milliliters.

23. The method of dispensing a semi-solid composition of claim 17, wherein the reset arrow is able to be manually, and bi-directionally aligned with the dosing knob volumetric markings while holding the barrel in position, while emitting an audible click with each reset dial actuation.

24. The method of dispensing a semi-solid composition of claim 17, further comprising the dispensing cap being colored to indicate an identity of the semi-solid composition that is stored within the barrel.

25. The method of dispensing a semi-solid composition of claim 17, wherein the overcap is childproof comprising a textured arrow 90 on an overcap sidewall 93 align-able with an opening 105 and an arrow 92 on the dispensing cap sidewall 104 in order for an adult to remove the overcap from the device.

26. The method of dispensing a semi-solid composition of claim 17, wherein the elevator platform further comprises: a vertical tube 85 housing the center threaded hole 84 in the center of the platform top side 81; and an elliptical side wall 82.

27. The method of dispensing a semi-solid composition of claim 26, wherein the elevator platform further comprises: an elliptical channel 80 on the top side 81 periphery, which is able to hold a drug backflow to form a liquid seal that prevents a drug leakage from the barrel.

28. The method of dispensing a semi-solid composition of claim 17, wherein the barrel further comprises a flat bottom end 51 with two concentric rings of: 1) outer protrusion members 50, and 2) inner protrusion members 54, extending vertically downward, said protrusions able to connect the barrel to the reset dial and the dosing knob, respectively.

29. The method of dispensing a semi-solid composition of claim 28, wherein the reset dial further comprises an inner wall 57 with a plurality of slots 52, wherein the outer protrusion members 50 fit into said slots enabling the reset dial to rotate bi-directional while the barrel and the dosing knob remain fixed.

30. The method of dispensing a semi-solid composition of claim 28, wherein the barrel inner protrusion members 54 extend from the barrel bottom end 51 through a reset dial circular cavity 60 and a dosing knob cavity 108 to connect without slippage to 5 of the 10 equidistant vertical grooves 91 on a dosing knob inner wall 73 to release a precise dose of the drug per each knob actuation.

31. The method of dispensing a semi-solid composition of claim 17, wherein the overcap is childproof comprising an outer textured arrow 90 on overcap sidewall 93 and an inner tab 100 behind arrow 90 that is align-able with an opening 105 and an arrow 92 on the dispensing cap sidewall 104 in order for an adult to remove the overcap from the device.

32. The method of dispensing a semi-solid composition of claim 17, wherein the overcap is reattached after administering said semi-solid composition by fitting two inner tabs 100 that are opposite said textured arrow 90, into a depression ring 103 under a ring 102 on the dispensing cap sidewall 104, wherein arrows 90 and 92 need not be aligned.

33. The method of dispensing a semi-solid composition of claim 17, wherein
the threaded spindle unit further comprises a connection unit with a solid tubular member 27 without threads and a circular end 26 positioned at the bottom end of screw 18, beneath the elevator platform 20;
the dosing knob further comprises a centered hollow channel 71 with a bottom hole 25; and
the solid tubular member 27 and the circular end 26 fit into the centered hollow channel 71 and the bottom hole 25 to connect the threaded spindle unit to the dosing knob.

34. The method of dispensing a semi-solid composition of claim 17, further comprising a system storing within the barrel and above the elevator platform, one or more doses of a semi-solid composition as shown in Table 1.
